(19) United States
(12) Patent Application Publication
(10) Pub. No.: US 2020/0001045 A1
(43) Pub. Date: Jan. 2, 2020

(54) CATHETER SYSTEM FOR CONTINUOUS IRRIGATION

(71) Applicant: InnoMed Two, L.L.C., Mobile, AL (US)

(72) Inventor: Matthew G. McIntyre, Theodore, AL (US)

(73) Assignee: InnoMedTwo, L.L.C., Mobile, AL (US)

(21) Appl. No.: 16/482,374

(22) PCT Filed: Apr. 6, 2017
(86) PCT No.: PCT/US2017/026450

§ 371 Date: Jul. 31, 2019

Related U.S. Application Data
(60) Provisional application No. 62/454,829, filed on Feb. 5, 2017.

(51) Int. Cl. A61M 25/00; A61M 39/10

(52) U.S. Cl. CPC A61M 25/0026; A61M 25/0017; A61M 25/0041; A61M 2025/0056; A61M 2025/0057; A61M 2210/1085; A61M 2210/1089; A61M 39/105

(57) ABSTRACT

An indwelling urinary catheter system having an elongated tubular catheter body 401 having a distal end and a proximal end; at least one sleeve portion 406 constructed out of a semi-permeable membranes surrounding at least one portion of the catheter body; at least one lumen to instill fluid into the catheter body; and a means to continuously efflux the instilled fluid through the semi-permeable membrane for circumferential egress of fluid out of the membrane around the catheter body. The catheter may further include a drainage lumen 414 extending through the catheter body from just short of the distal end to the proximal end and an opening or eyelet 405 in the catheter body just short of the distal end of the catheter body to permit urine to drain from a patient's bladder into the drainage lumen. A retaining mechanism may also be comprised.

### RELATED APPLICATIONS

This application claims priority to U.S. Provisional Application No. 62/454,829 filed Feb. 5, 2017. The entire contents of the above application are hereby incorporated by reference as though fully set forth herein.

### FIELD

The present invention pertains to a catheter, and more particularly, to intra-urethral or indwelling catheters capable of effluxing fluids.

### BACKGROUND

The traditional Foley-type catheter is well known in the art and comprises an inflatable balloon disposed within the patient's bladder and a discharge tube extending through the urethra to the exterior. The Foley-type catheter provides passive urinary drainage, and the ability to clamp the catheter closed at a location exterior of the patient.

Urethral catheters, such as Foley-catheters, are used to drain urine from the bladder. A urinary tract infection (also called “UTI”) is an infection in the urinary system, which includes the bladder and kidneys. When a urinary catheter is inserted into the bladder, germs can migrate along the catheter and cause an infection in the bladder or kidney; resulting in a catheter-associated urinary tract infection (or “CAUTI”). CAUTIs are the most common of hospital-acquired infections. In fact, 40% of all nosocomial infections and over 100,000 admissions to hospital within the USA annually are attributable to CAUTIs.¹ Outcomes associated with CAUTIs include bacteremia and sepsis. While morbidity that is attributable to a single episode of catheterization is limited, the high frequency of catheter use (around 25% of hospitalized patients) means that the cumulative burden of CAUTIs on patients and hospitals is substantial.² ¹ D. Cardo et al. National Nosocomial Infections Surveillance (NNIS) System Report, data summary from January 1992 through June 2004, issued October 2004. Am. J. Infect. Control, 32 (2004), pp. 470-485.² Lo, E. et al. (2008). Strategies to Prevent Catheter-Associated Urinary Tract Infections in Acute Care Hospitals. Infection Control and Hospital Epidemiology, 29(S1), S41-S50. doi:10.1086/591066

When sterile urinary catheters are inserted into the bladder, components in urine, blood, or surrounding tissue, such as polysaccharides, ions, and glycoproteins, are deposited on the surface of the device allowing the formation of biofilms. Biofilms are highly structured and actively growing bacterial communities that consist of multiple bacterial layers protected by a thick exopolysaccharide layer³. Biofilms are resistant to antibiotics/antimicrobials due to the fact that these agents cannot penetrate sufficiently through the exopolysaccharide layer. ³ Tenke, P.; Koves, B.; Nagy, K.; Hultgren, S. J.; Mendling, W.; Wullt, B.; Grabe, M.; Wagenlehner, F. M.; Cek, M.; Pickard, R.; et al. Update on biofilm infections in the urinary tract. World J. Urol. 2012, 30, 51-57.

According to Centers for Disease Control and Prevention (CDC), there was no change in overall catheter-associated urinary tract infections (CAUTI) rates between 2009 and 2014. (see https://www.cdc.gov/hai/surveillance/). This is not surprising, as while a variety of approaches for prevention of biofilm formation include the use of biocoatings, impregnating materials with antibiotics, antimicrobials or other materials as well as catheters capable of eluting antibiotics and/or antimicrobials have been used, none have been fully effective. Further, one of the major complications associated with antibiotic based coatings is the development of resistance. For example, one approach has been to attach active biocides such as antibiotics to biomaterial surfaces, or to impregnate them into the biomaterial itself by coating device surfaces or impregnating device surfaces with antibiotics such as ciprofloxacin, gentamicin, norfloxacin, and nitrofurazone. When used in clinical studies, the uncontrolled release profiles of the drugs resulted in the elution of initial high local concentrations that may initially damage the cells followed by concentrations that are not inhibitory.⁴ By not killing all of the bacteria effectively, any subsequent infection will be more difficult to eradicate due to the development of resistance. ⁴ Walder, B.; Pittet, D.; Tramer, M. R. Prevention of bloodstream infections with central venous catheters treated with anti-infective agents depends on catheter type and insertion time: Evidence from a meta-analysis. Infect. Control Hosp. Epidemiol. 2002, 23, 748-756.

Looking at the physiology of the urethra, UTIs are generally avoided because the act of urination (voiding) flushes everything, including bacteria. Further, there are glands in urethra that secretes protecting mucus. Several drug eluting urinary catheters are known in the prior art. Drug-eluting urinary catheters generally consist of three parts—the catheter tube, a polymer coating that binds the drug to the tube and releases the drug. The drug is slowly and continuously released into the bladder or along urethra; however, there is no continual washing of the periurethral space, where bacteria adhere, form biofilms and result in bacterial infections.

It would therefore be useful to magnify the effect of the glands in the urethra that protect from infection in the context of catheters.

### BRIEF SUMMARY OF THE INVENTION

It is therefore one object of the present invention to provide an indwelling urinary catheter system having (1) an elongated tubular catheter body having a distal end and a proximal end; (2) at least one sleeve portion constructed substantially out of a semipermeable membranes surrounding at least one portion of the catheter body; (3) at least one lumen to instill fluid into the catheter body; and (4) a means to continuously efflux the instilled fluid through the semipermeable membrane of at least one sleeve resulting in the circumferential egress of fluid out of the semipermeable membrane around the catheter body. The catheter may further include a drainage lumen extending through the catheter body from just short of the distal end to the proximal end and an opening or eyelet in the catheter body just short of the distal end of the catheter body to permit urine to drain from a patient's bladder into the drainage lumen. The catheter body is disposed within the urethra of the patient and a retaining mechanism, such as an inflatable balloon, is disposed within the patient's bladder to retain the catheter in position. The fluid instilled into the catheter body and effluxed from the sleeve portion(s) may include, but is not limited to, antiseptics, antibiotics or antimicrobials, and/or combinations thereof to prevent biofilm formation on the exterior surface of the catheter body. The fluid may also include certain therapeutic agents used in intravesical therapy, such as immunotherapy agents or chemotherapeutic agents. The fluid may also include agents for patient comfort, such as antispasmodics and pain medicines. All such agents can be effluxed directly into the bladder through the semipermeable sleeve portion around the catheter tip placed within the bladder.

It is another object of the present invention to provide different embodiments of the urinary catheter system that match the particular anatomical characteristics of a patient with respect to male or female anatomy. For example, a retention collar may be positioned on the catheter body for female patients or a space may be provided for the prostate for male patients.

### BRIEF DESCRIPTION OF THE DRAWINGS

FIG. 1 is cross section view of a traditional catheter for insertion into the bladder.

FIG. 2 is a front perspective view of a traditional 2-way urinary catheter.

FIG. 3 is a front perspective view of a traditional 3-way urinary catheter with a cutaway cross section of the catheter body.

FIG. 4A is a front perspective view of one embodiment of the urinary catheter of the present invention with a cutaway cross section of the catheter body.

FIG. 4B is a front perspective view of one embodiment of the urinary catheter of the present invention with a cutaway cross section of the sleeve section.

FIG. 5A is a front perspective view of an alternative embodiment of the urinary catheter of the present invention with a cutaway cross section of the catheter body.

FIG. 5B is a front perspective view of an alternative embodiment of the urinary catheter of the present invention with a cutaway cross section of the sleeve.

FIG. 6A is a front perspective view of an alternative embodiment of the urinary catheter of the present invention with a cutaway cross section of the catheter body.

FIG. 6B is a front perspective view of an alternative embodiment of the urinary catheter of the present invention with a cutaway cross section of the sleeve.

FIG. 7A is a cross section view of the placement of a catheter in a male.

FIG. 7B is a cross section view of the placement of a catheter in a female.

FIG. 8A is a front perspective view of one embodiment of the present invention for use in female patients.

FIG. 8B is a front perspective view of one embodiment of the present invention for use in female patients with a cutaway cross section of the sleeve.

FIG. 9A is a front perspective view of one embodiment of the present invention for use in male patients.

FIG. 9B is a front perspective view of one embodiment of the present invention for use in male patients with a cutaway cross section of the sleeve.

FIG. 10A is a front perspective view of one embodiment of the present invention with a couvelaire tip.

FIG. 10B is a front perspective view of one embodiment of the present invention with a dufour tip.

FIG. 10C is a front perspective view of one embodiment of the present invention with a coude tip.

FIG. 11A is a front perspective view of an alternative embodiment of the present invention with a couvelaire tip.

FIG. 11B is a front perspective view of an alternative embodiment of the present invention with a dufour tip.

FIG. 11C is a front perspective view of an alternative embodiment of the present invention with a coude tip.

FIG. 12A is a front perspective view of an alternative embodiment of the present invention with a couvelaire tip.

FIG. 12B is a front perspective view of an alternative embodiment of the present invention with a dufour tip.

FIG. 12C is a front perspective view of an alternative embodiment of the present invention with a coude tip.

### DETAILED DESCRIPTION

For the purposes of the present invention, the term “semipermeable” is intended to encompass not only those materials that are semipermeable by their nature (i.e. those that allow certain substances to pass through it while not allowing other materials to pass through it) but materials that may be made semipermeable by creating pores of a predetermined size that would allow certain substances to pass through it while not allowing other materials to pass through it.

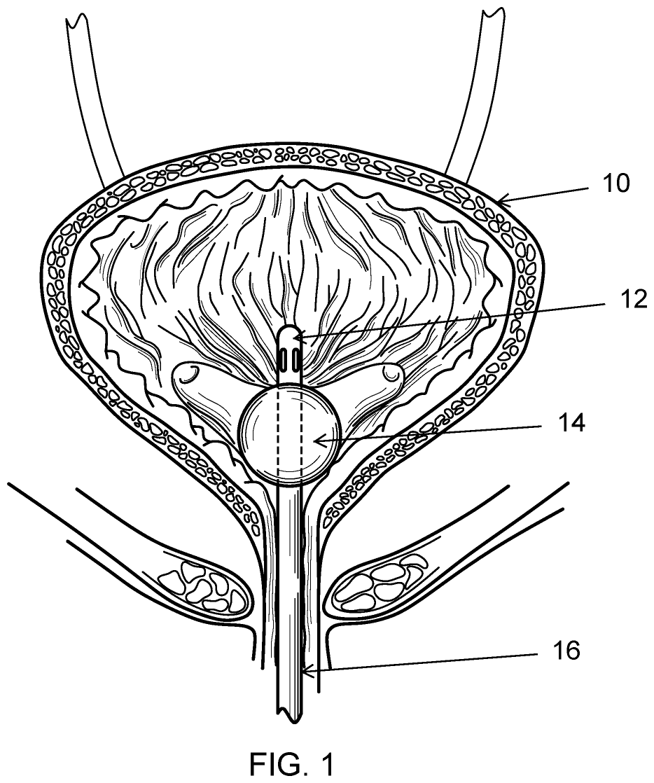

Turning to the drawings, there shown in FIG. 1 is a traditional catheter for insertion into a cavity, duct, or a vessel to permit injection or withdrawal of fluids into or from the cavity, duct, or vessel, or to establish patency of a passageway. For example, the catheter body 16 may be inserted through a patient's urethra and into the patient's bladder 10 for draining urine from the bladder and/or instilling fluid into the bladder through slots in the tip 12 of the catheter. A retaining device, such as the balloon 14, is used to maintain placement of the catheter in the bladder.

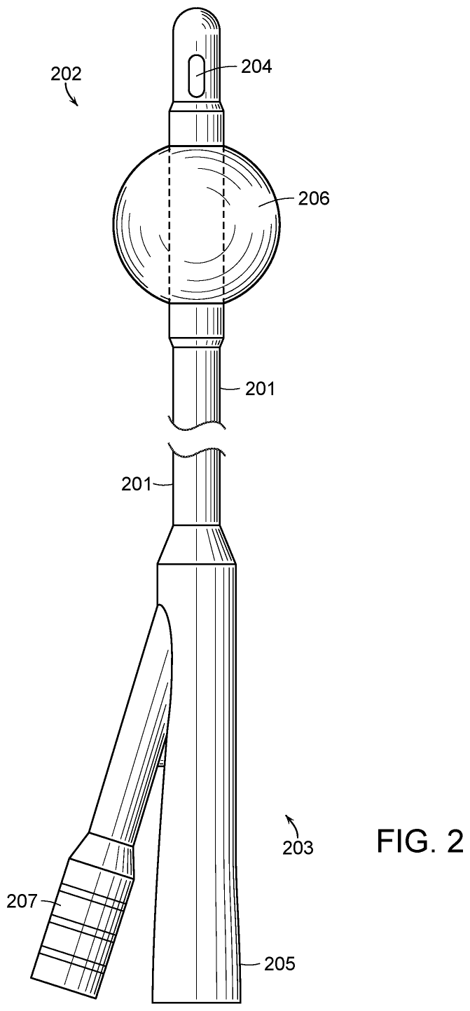

Turning to FIG. 2, a traditional 2-way urinary catheter is represented with a catheter body 201 having a distal end 202 and a proximal end 203 with the catheter body 201 connecting an opening or eyelet 204 at the distal end 202 to a drainage lumen 205 at the proximal end 203 of the catheter body 201 through which fluid may flow into the drainage lumen 205 when the catheter is used to drain fluid from the bladder. An inflatable tube section 206 with an inflation lumen 207 extends along the length of the catheter body 201 and communicates with the inflatable tube section 206. Inflation fluid, such as distilled water, is passed through inflation lumen 207 into the tube section 206 to inflate the tube section 206, and the inflation fluid is withdrawn from the tube section 206 into and through the inflation lumen 207 when it is desired to deflate the tube section 206.

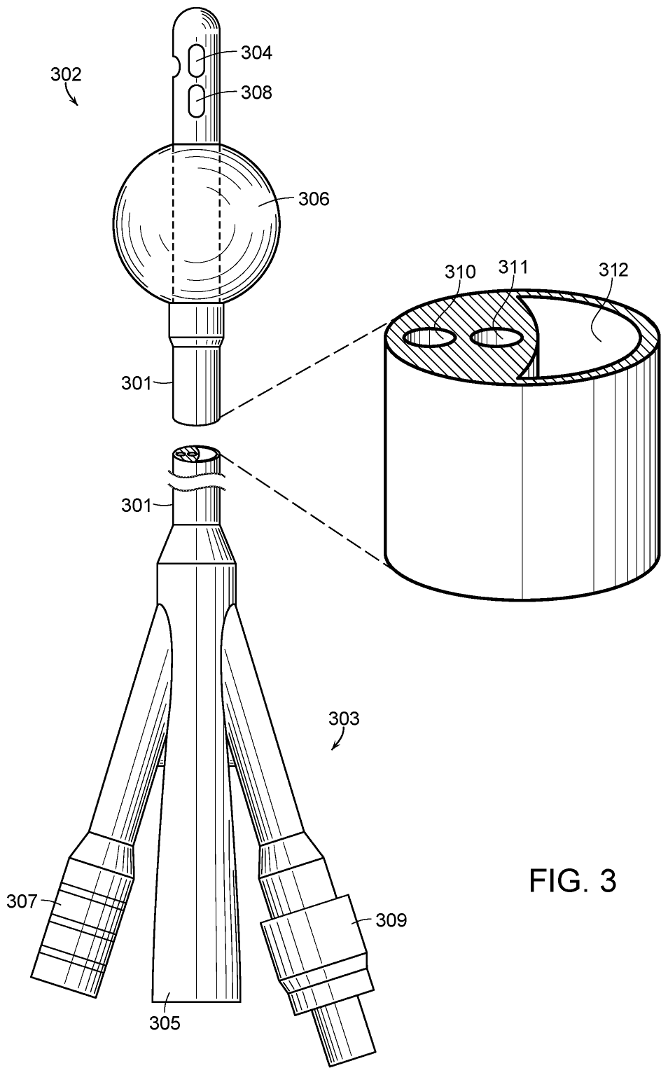

Turning to FIG. 3, a traditional 3-way urinary catheter is represented that is essentially the same as the catheter shown in FIG. 2, except it includes an instillation lumen 309 that extends from the catheter body 301 at the proximal end 303. The fluid instilled into the catheter body 301 is passed through tube 311 in the catheter body 301 and into the bladder through the opening or eyelet 304 and then the fluid is subsequently drained through the opening or eyelet 308 through tube 312 in the catheter body 301 and out the drainage lumen 305. As shown in the cross section, the fluid instilled into the catheter body 301 passes through tube 311 in the catheter body. Inflation fluid is passed through inflation lumen 307 and through tube 310 to inflate the tube section 306. Fluid that is drained through eyelet 308 at the distal end 302 passes through tube 312 and out the drainage lumen 305.

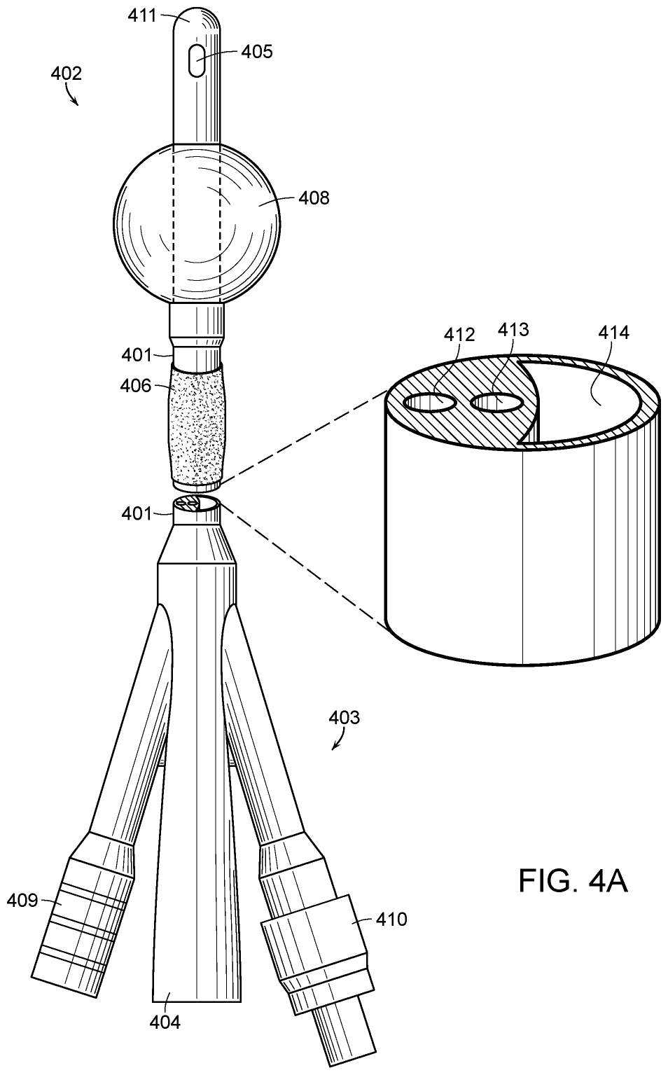

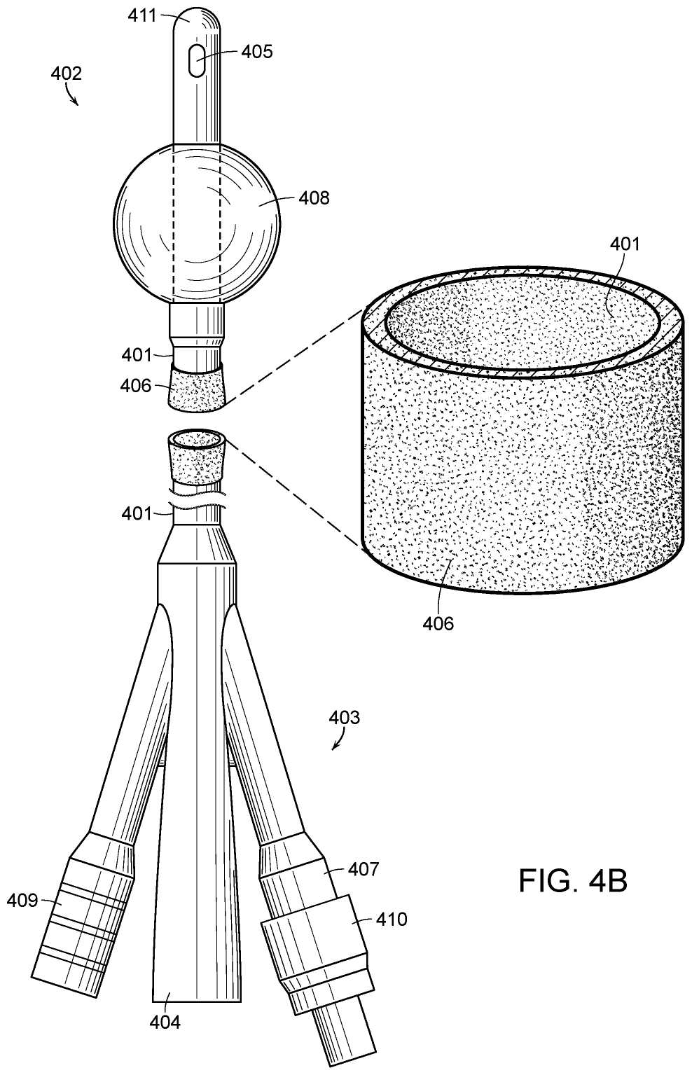

Referring to FIG. 4A, the catheter of the present invention includes an elongated tubular catheter body 401 having a distal end 402 and a proximal end 403. A drainage lumen 404 extends through tube 414 in the catheter body 401 from the distal end 402 to the proximal end 403. The drainage lumen 404 communicates with an opening or eyelet 405 in the catheter body 401 at the distal end 402 of the catheter body 401 through which the fluid may flow into the drainage lumen 404 when the catheter is used to drain a fluid from a cavity, duct, or vessel (e.g., draining urine from a person's bladder). A sleeve portion 406 constructed from a semipermeable membrane is formed over the catheter body 401. An instillation lumen 410 extends from the catheter body 401 at the proximal end 403. The instillation lumen 410 connects with the sleeve portion 406 using tube 413 that runs through the length of the catheter body 401. The fluid instilled into the catheter body 401 through the tube 413 is continuously effluxed from the sleeve portion 406 through the semipermeable membrane in a circumferential controlled delivery to continuously irrigate the periurethral space and the catheter body 401 to prevent formation of biofilm and further ensuing bacterial infection. The fluid may include, but is not limited to, antiseptics, antibiotics or antimicrobials and/or combinations thereof to prevent biofilm formation on the exterior surface of the catheter body. Inflation fluid is passed through inflation lumen 409 and through tube 412 in the catheter body 401 to inflate the tube section 408.

Turning to FIG. 4B, a cross section cutaway of the sleeve portion 406 illustrates that the sleeve circumferentially surrounds the catheter body 401. In the preferred embodiment, the sleeve 406 is manufactured as a continuous part over the catheter body 401. It may be secured to the catheter body 401 using methods known in the art such as adhesive attachment or heat press melting. Additionally, the sleeve 406 is preferably constructed from a non-elastic material to allow the effluxed fluid to irrigate the periurethral space without putting pressure on the urethra. In the preferred embodiment, the fluid effluxed from the sleeve 406 exits through the urethral opening and may be collected by a sponge or padded surface. Ideally around 300-500 mL of fluid a day would be effluxed resulting in a collection rate in the sponge or padded surface of about 20 ccs per hour. This is manageable in a hospital care setting with intermittent replacement of the sponge or padded surface.

Referring to FIG. 4A, the preferred embodiment a retaining mechanism near the distal end 402 of the catheter body 401 is generally an inflatable tube section 408 with an inflation lumen 409 that extends the length of the catheter body 401 through tube 412 and communicates with the inflatable tube section 408. Inflation fluid, such as distilled water, is passed through inflation lumen 409 into the tube section 408 to inflate the tube section 408, and the inflation fluid is withdrawn from the tube section 408 into and through the inflation lumen 409 when it is desired to deflate the tube section 408. When the inflatable tube section 408 is not inflated, it lies substantially parallel along the central axis of the catheter body 401, forming a cylinder having a diameter that substantially matches the outer diameter of the catheter body 401.

The fluid instilled into the catheter body 401 and effluxed out of the semipermeable membrane sleeve 406 of the catheter body may be pushed through the device using various mechanisms, including but not limited to, a pressure and flow regulating valve to control rate of flow for a specific fluid at a specific pressure that is installed at the effluxing instillation lumen 410 or using a pump tension device, such as a plastic ball that is blown up and then pushes fluid out at a constant rate. It is also contemplated that an intravenous (IV) pump operating at a continuous rate may also be used to move fluid through the instillation lumen 410 and out of the semipermeable membrane of the sleeve portion 406. Again, the rate would be predetermined based on the semipermeable membrane material as well as the molecular weight cut off (MWCO) of the agent instilled into the catheter and effluxed through the semipermeable membrane to ensure that the agent is being pushed with sufficient pressure and at a sufficient rate to effectively continuously wash the periurethral space around the catheter body 401.

It is further contemplated that a drug eluting portion could be located within the tip 411 of catheter body 401 that goes into the bladder that could be used to deliver drugs to the bladder itself, such as an antispasmodic, pain medicines, antibiotics, antiseptics, antimicrobials and combinations thereof.

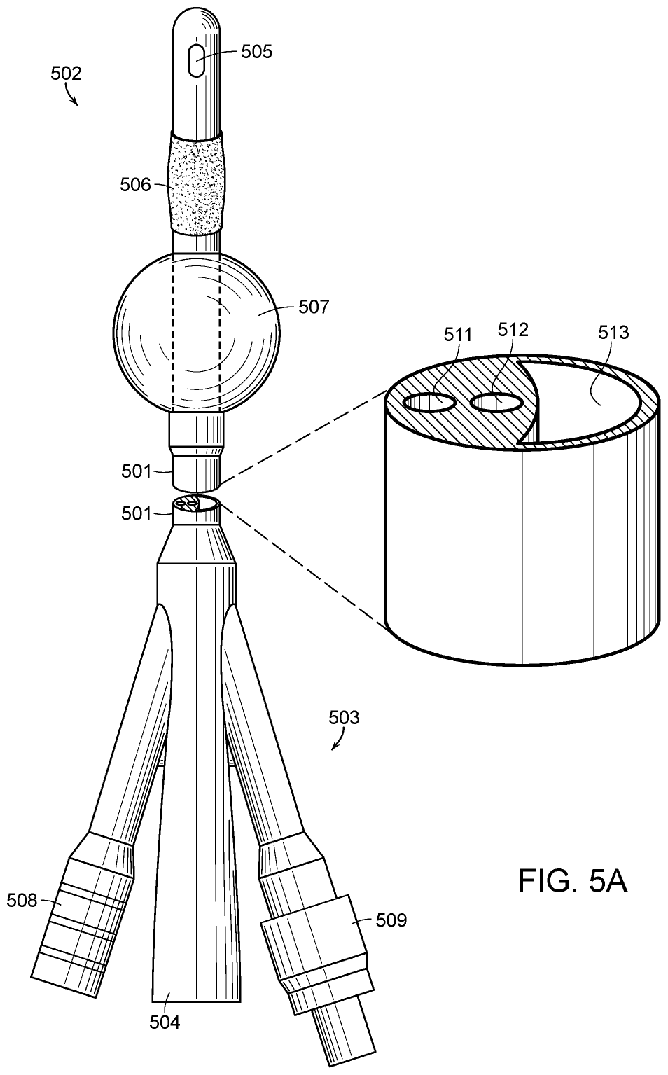

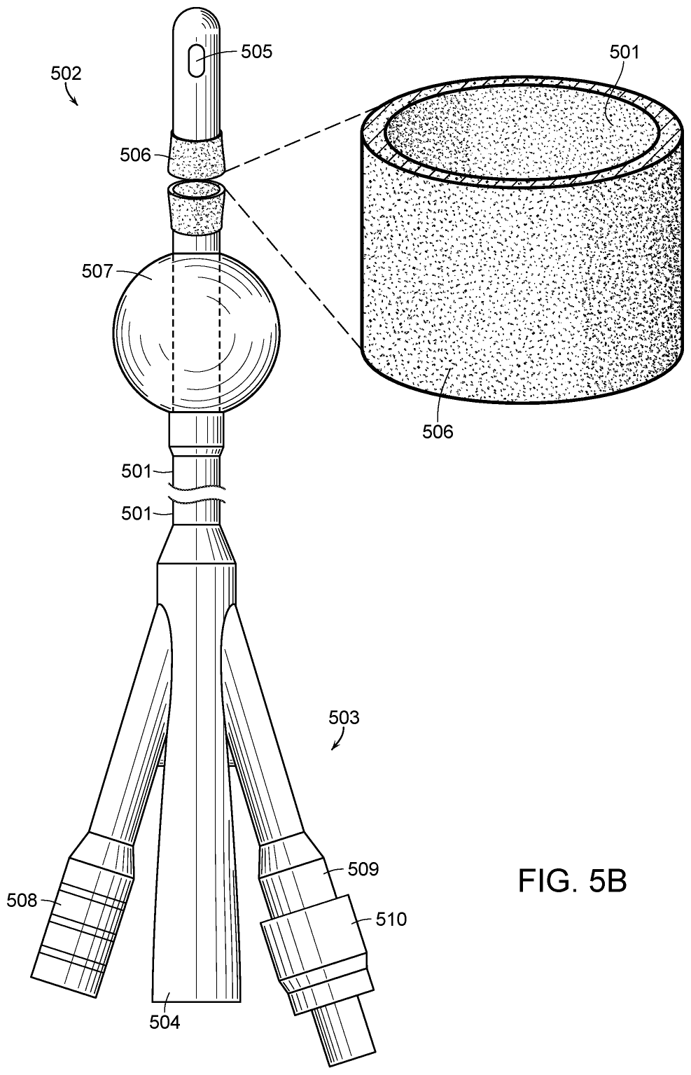

Turning to FIG. 5A, an alternative embodiment of the present invention is represented with an elongated tubular catheter body 501 having a distal end 502 and a proximal end 503. A drainage lumen 504 extends through tube 513 in the catheter body 501 from the distal end 502 to the proximal end 503, and the drainage lumen 503 communicates with an opening or eyelet 505 in the catheter body 501 at the distal end 502 of the catheter body 501 through which the fluid may flow into the drainage lumen 504 when the catheter is used to drain a fluid from a cavity, duct, or vessel (e.g., draining urine from a person's bladder). The retaining mechanism in this example is an inflatable tube section 507 with an inflation lumen 508 that extends though the length of the catheter body 501 though tube 511 and communicates with the inflatable tube section 507. Inflation fluid, such as distilled water, is passed through inflation lumen 508 into the tube section 507 to inflate the tube section 507, and the inflation fluid is withdrawn from the tube section 507 into and through the inflation lumen 508 when it is desired to deflate the tube section 507. When the inflatable tube section 507 is not inflated, it lies substantially parallel along the central axis of the catheter body 501, forming a cylinder having a diameter that substantially matches the outer diameter of the catheter body 501.

A sleeve portion 506 constructed from a semipermeable membrane is formed over the catheter body 501 above the tube section 507. An instillation lumen 509 extends from the catheter body 501 at the proximal end 504. The instillation lumen 509 connects with the sleeve portion 506 using tube 512 that runs through the length of the catheter body 501. The fluid instilled into the catheter body 501 through the tube is continuously effluxed from the sleeve portion 506 through the semipermeable membrane and into the bladder.

Turning to FIG. 5B, a cross section cutaway of the sleeve portion 506 illustrates that the sleeve circumferentially surrounds the catheter body 501. In the preferred embodiment, the sleeve 506 is manufactured as a continuous part over the catheter body 501. It may be secured to the catheter body 501 using methods known in the art such as adhesive attachment or heat press melting. The fluid effluxed through the sleeve 506 includes, but is not limited to, certain therapeutic agents used in intravesical therapy, such as immunotherapy agents or chemotherapeutic agents, as well as antispasmodic agents and numbing agents such as lidocaine. The semipermeable membrane of the sleeve 506 allows certain substances to pass through it but not others, such as allowing fluids to efflux out of the sleeve 506 but not allowing bacteria or other contaminants into the sleeve 506. The semipermeable membrane also allows the use of a small amount of fluid everywhere circumfrentially along the length of the catheter body portion in the bladder as well as into the bladder space. The pore size of the semipermeable membrane is predetermined based on the agent instilled into the catheter and effluxed from the semipermeable membrane to ensure that the agent may pass through the semipermeable membrane of the sleeve 506 and may be effluxed with sufficient pressure and at a sufficient rate to effectively continuously wash the bladder with the fluid. This method is a superior mechanism to deliver therapies such as antispasmodic agents and numbing agents than an instillation performed using a traditional catheter. With a traditional catheter, instillations are performed on an intermittent basis wherein the medicine is delivered through a single lumen catheter and then removed. The patient then voids the bladder to remove the medicine. The present invention allows the medicine to be slowly effluxed into the bladder at a continuous rate. This is especially useful after transurethral surgery on a patient. The catheter of the present invention can be placed shortly after surgery so that a drug, such as an antispasmodic or pain medication, may be effluxed from the sleeve 506 for the next four to six hours, resulting in steady patient pain and discomfort management.

The fluid instilled into the catheter body and effluxed out of the semipermeable membrane of the sleeve portion 506 over the catheter body 501 and into the bladder may be pushed through the device using various mechanisms, including but not limited to, a pressure and flow regulating valve to control rate of flow for a specific fluid at a specific pressure that is installed at the effluxing instillation lumen port 510 or using a pump tension device, such as a plastic ball that is blown up and it then pushes fluid out at a constant rate. It is also contemplated that an intravenous (IV) pump operating at a continuous rate may also be used to move fluid through the instillation lumen and out of the semipermeable membrane of the sleeve portion 506. Again, the rate would be predetermined based on the agent instilled into the catheter and effluxed from the semipermeable membrane to ensure that the agent is being pushed with sufficient pressure and at a sufficient rate to effectively continuously wash the bladder space.

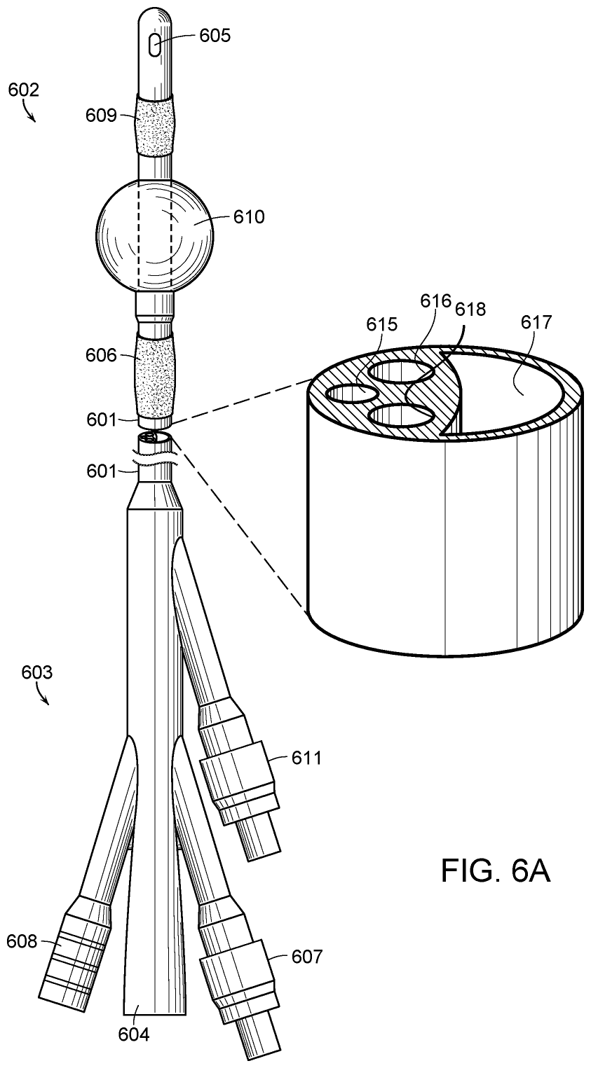

Turning to FIGS. 6A-B, another embodiment of the present invention uses both sleeve portions of FIGS. 4-5. This results in a 4 way catheter capable of both effluxing fluid to continuously irrigate the periurethral space as well as effluxing fluid to continuously wash the bladder space.

As shown in FIG. 6A an elongated tubular catheter body 601 having a distal end 602 and a proximal end 603. A drainage lumen 604 extends through tube 617 in the catheter body 601 from the distal end 602 to the proximal end 603, and the drainage lumen 604 communicates with an opening or eyelet 605 in the catheter body 601 at the distal end 602 of the catheter body 601 through which the fluid may flow into the drainage lumen 604 when the catheter is used to drain a fluid from a cavity, duct, or vessel (e.g., draining urine from a person's bladder). A first sleeve portion 606 constructed from a semipermeable membrane is formed over the catheter body 601. An instillation lumen 607 extends from the catheter body 601 at the distal end 602. The instillation lumen 607 connects with the first sleeve portion 606 using tube 616 that runs through the length of the catheter body 601. The fluid instilled into the catheter body 601 through the tube is continuously effluxed from the sleeve portion 606 through the semipermeable membrane in a circumferential controlled delivery to continuously irrigate the periurethral space and the catheter body 601 to prevent formation of biofilm and further ensuring bacterial infection. The fluid may include, but is not limited to, antiseptics, antibiotics or antimicrobials and/or combinations thereof to prevent biofilm formation on the exterior surface of the catheter body.

A second sleeve portion 609 constructed from a semipermeable membrane is formed over the catheter body 601 above the tube section 610. An instillation lumen 611 extends from the catheter body 601 at the distal end 602. The instillation lumen 611 connects with the sleeve portion 609 using tube 618 that runs through the length of the catheter body 601 The fluid instilled into the catheter body 601 through the tube 618 is continuously effluxed from the sleeve portion 609 through the semipermeable membrane and into the bladder itself.

The fluid effluxed through the sleeve 609 includes, but is not limited to, certain therapeutic agents used in intravesical therapy such as immunotherapy agents or chemotherapeutic agents, antispasmodic agents and numbing agents, such as lidocaine.

The fluid instilled into the catheter body and effluxed out of the semipermeable membrane of the sleeve portions 606 and 609 may be pushed through the device using various mechanisms, including but not limited to, pressure and flow regulating valves to control rate of flow for a specific fluid at a specific pressure that is installed at the effluxing instillation lumen ports 607 and 611, or using a pump tension device, such as a plastic ball that you blow up and it then pushes fluid out at a constant rate. It is also contemplated that an intravenous (IV) pump operating at a continuous rate may also be used to move fluid through the instillation lumens 607 and 611 and out of the semipermeable membrane of the sleeve portions 606 and 609, respectively. Again, the rate would be predetermined based on the agent instilled into the catheter and effluxed from the semipermeable membrane to ensure that the agent is being pushed with sufficient pressure and at a sufficient rate to effectively continuously wash the periuretheral and bladder spaces.

Turning to FIG. 6B, a cross section cutaway of the sleeve portions 606 and 609 illustrates that the sleeve circumferentially surrounds the catheter body 601. In the preferred embodiment, the sleeve portions 606 and 609 are manufactured as continuous parts over the catheter body 601. They may be secured to the catheter body 601 using methods known in the art such as adhesive attachment or heat press melting.

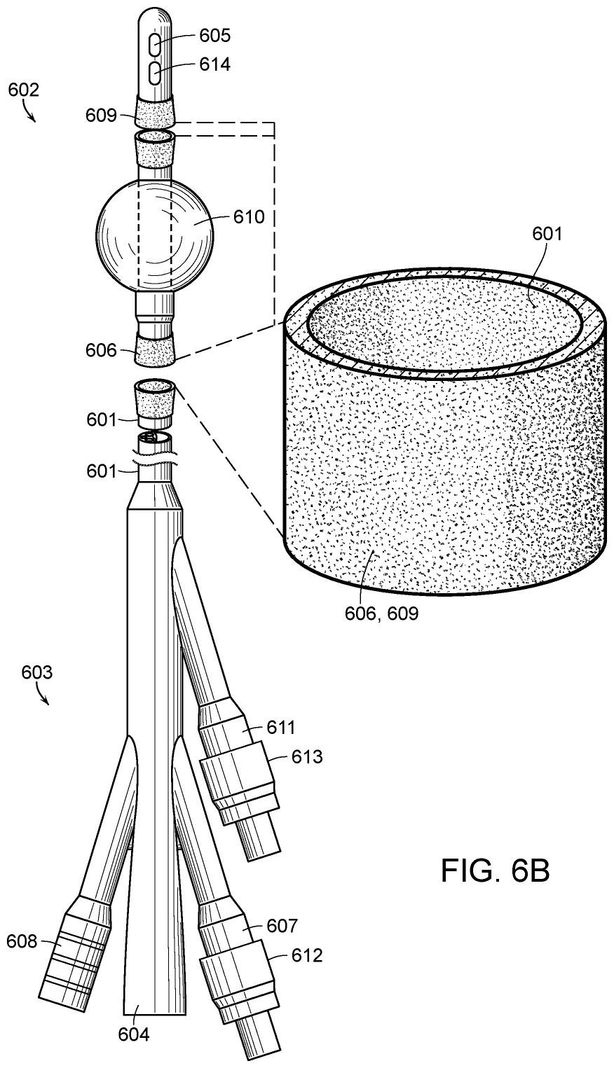

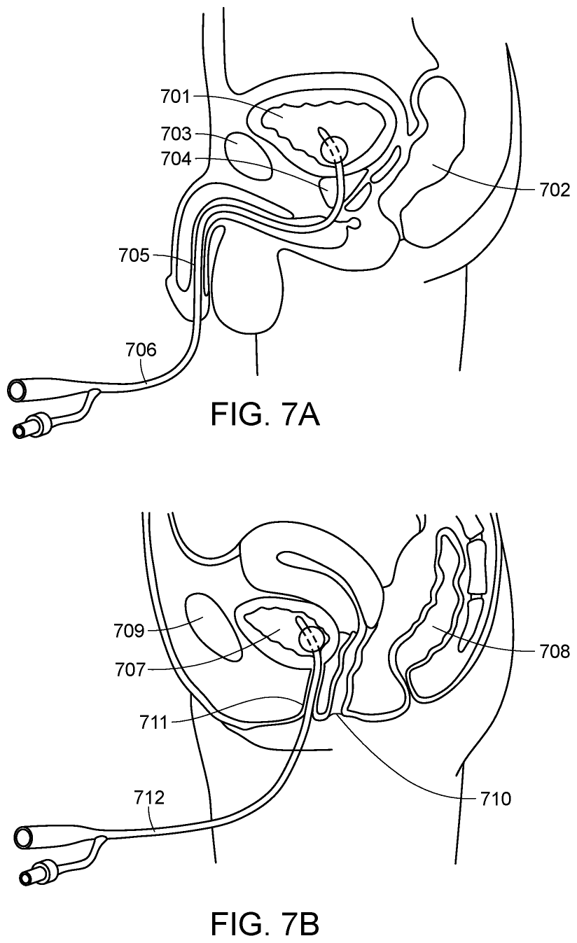

Turning to FIG. 7A-B, the differences in anatomy for the placement of a urinary catheter are shown. The male anatomy of FIG. 7A results in a larger portion of the catheter body in the periurethral space than the female counterpart. FIG. 7A shows the bladder 701, rectum 702, pubic bone 703, prostate 704, urethra 705 and the catheter 706. The catheter 706 must also be fed past the prostate 704 in males before it can be retained in the bladder 701. The female anatomy of FIG. 7B results in a shorter portion of the catheter body needed to fill the periurethral space. FIG. 7B shows the bladder 707, rectum 708, pubic bone 709, vagina 710, urethra 711 and catheter 712.

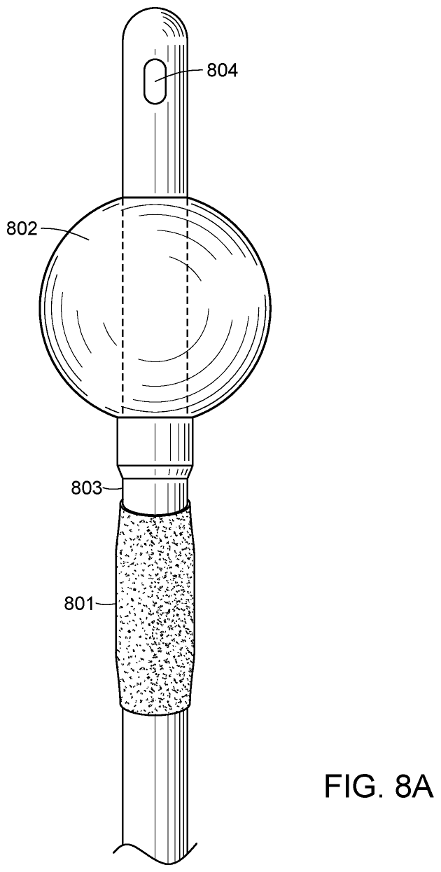

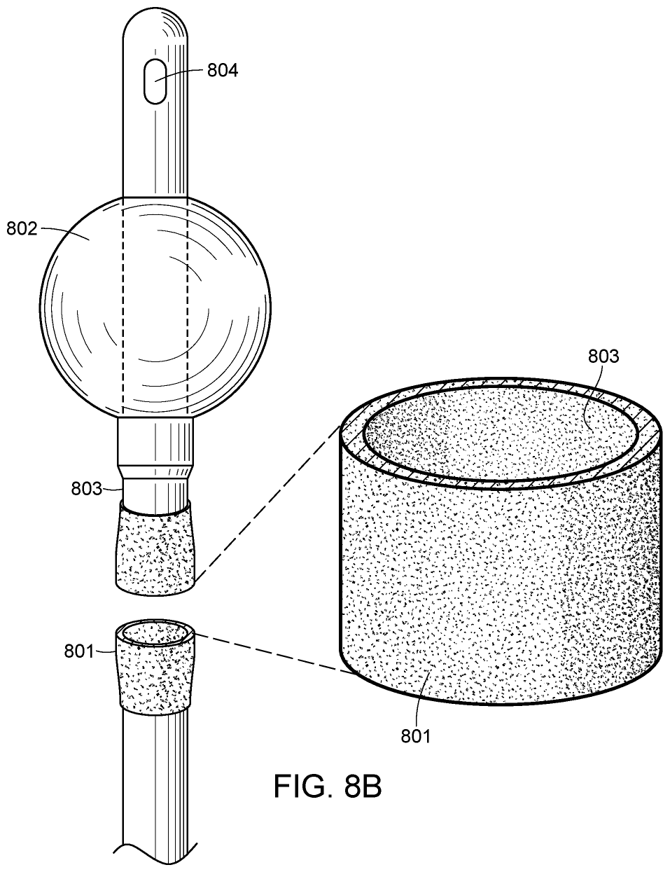

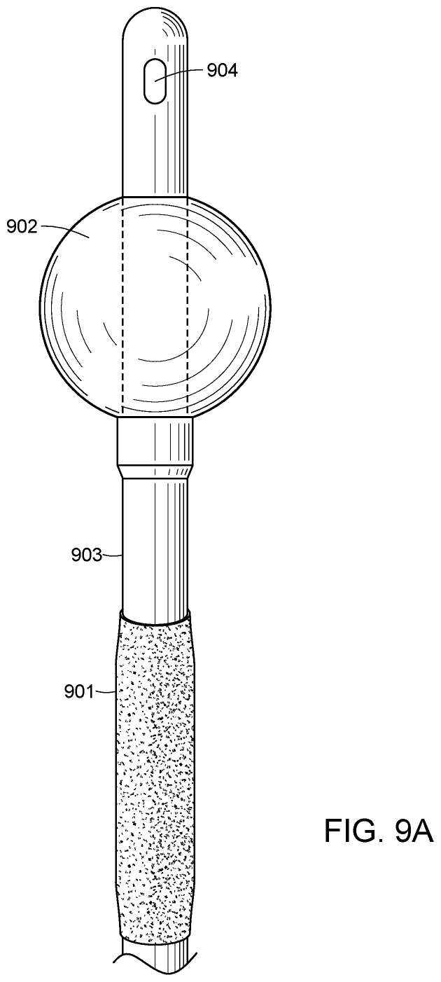

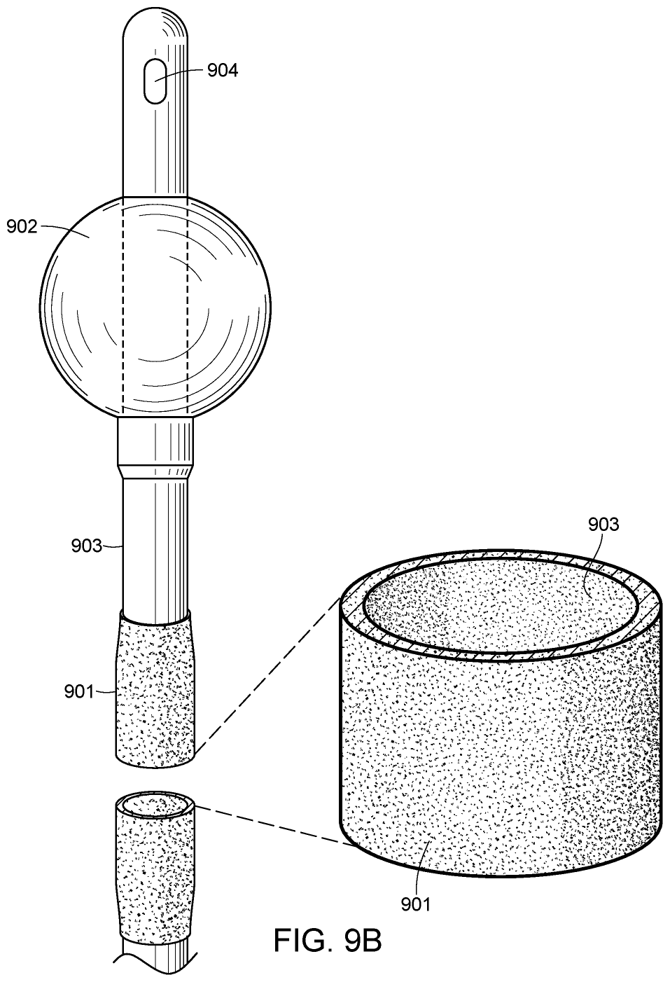

Taking these anatomical differences into consideration, FIG. 8A-B shows the distal end of the catheter of FIG. 4 as used for female anatomy whereas FIG. 9A-B shows the distal end of the catheter of FIG. 5 as used for male anatomy. The sleeve portion 801 of FIG. 8A-B is shorter than the sleeve portion 901 of FIG. 9A-B. Additionally, there is a larger space 903 between the sleeve portion 901 and the inflatable portion 902 than the space 803 between the sleeve portion 801 and the inflatable portion 802, which accommodates placement of the catheter in the presence of the prostate.

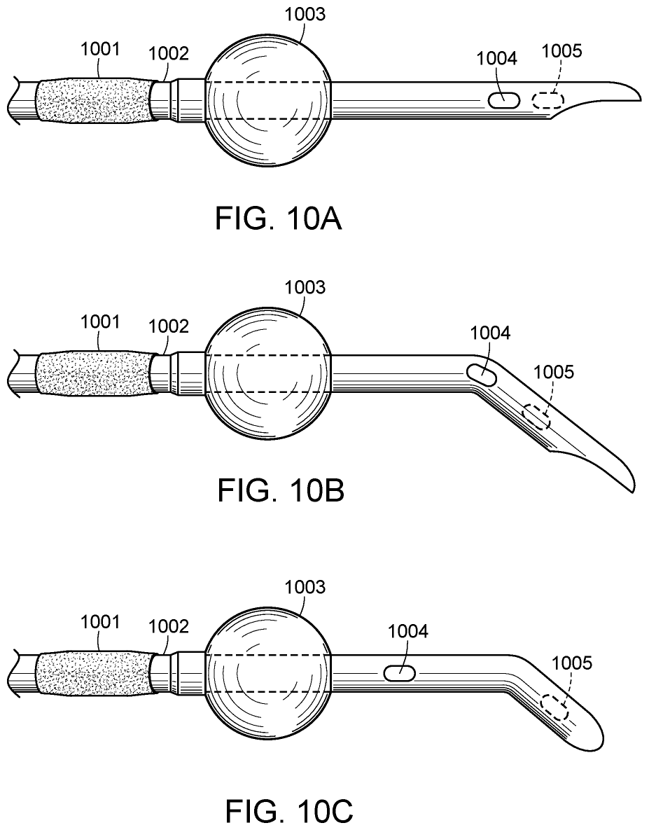

As shown in FIGS. 10A-C, one embodiment of the invention shown in FIGS. 4A-B with sleeve portion 1001, catheter body 1002, retaining device 1003, drainage eyelet 1004 and alternative instillation eyelet 1005 may have various shapes to the end that is inserted into the bladder. For example, FIG. 10 A shows a couvelaire tip, FIG. 10B shows a dufour tip and FIG. 10C shows a coude tip.

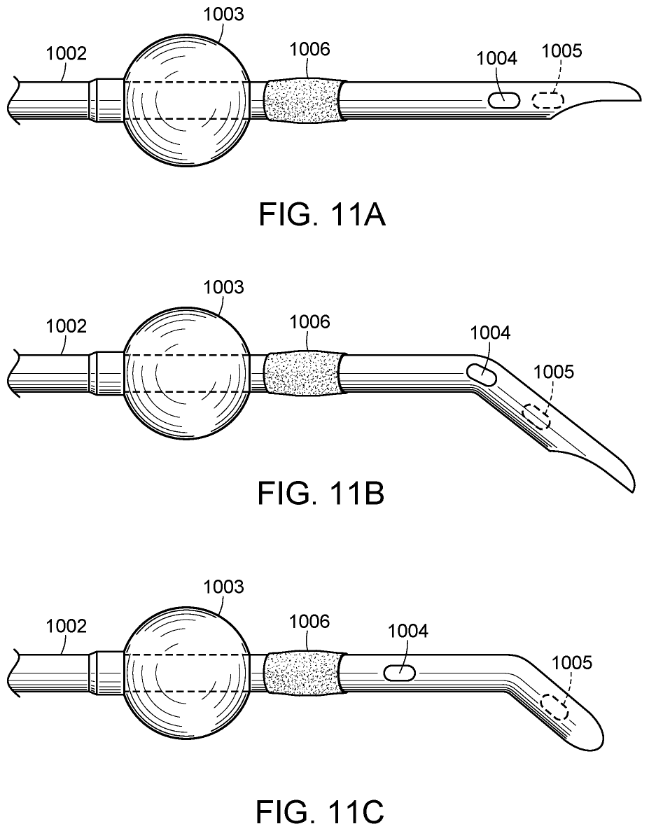

As shown in FIGS. 11A-C, one embodiment of the invention shown in FIGS. 5A-B with sleeve portion 1006, catheter body 1002, retaining device 1003, drainage eyelet 1004 and alternative instillation eyelet 1005 may have various shapes to the end that is inserted into the bladder. For example, FIG. 11 A shows a couvelaire tip, FIG. 11B shows a dufour tip and FIG. 11C shows a coude tip.

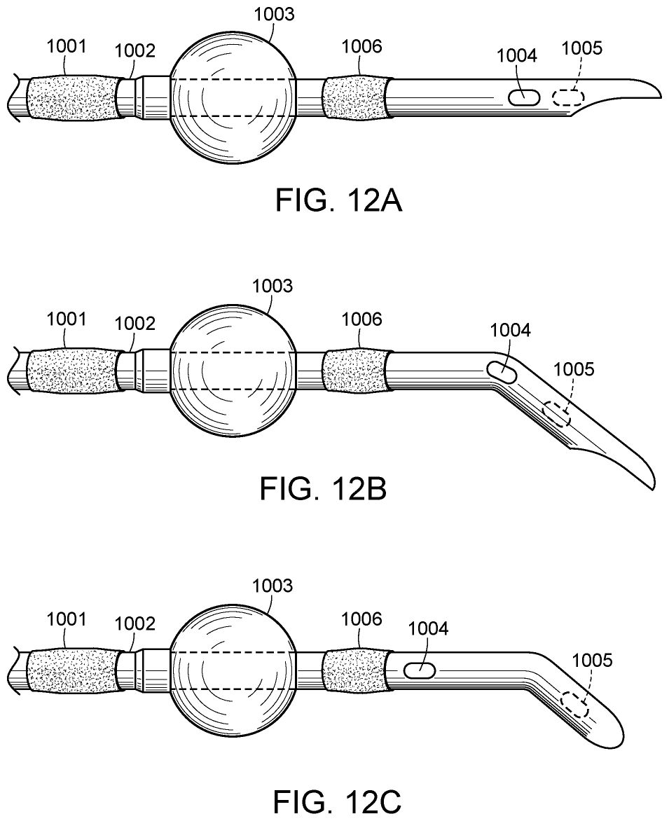

As shown in FIGS. 12A-C, one embodiment of the invention shown in FIGS. 6A-B with sleeve portions 1001 and 1006, catheter body 1002, retaining device 1003, drainage eyelet 1004 and alternative instillation eyelet 1005 may have various shapes to the end that is inserted into the bladder. For example, FIG. 12 A shows a couvelaire tip, FIG. 12B shows a dufour tip and FIG. 12C shows a coude tip.

It is necessary for the fluid to be effluxed continuously at a basal rate to effect the continual washing of the periurethral space, where bacteria adhere, to prevent formation of biofilms and resulting bacterial infections. However, it is also contemplated that the fluid may be continuously effluxed from the semipermeable membrane(s) in a peristaltic wave action along the length of the catheter body in addition to the basal rate.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the method (and components of the individual operating components of the method) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections might be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

1-33. (canceled)

34. A urinary catheter system comprising:
an elongate catheter body, the elongate catheter body comprising an instillation lumen disposed within and extending through said catheter body, the instillation lumen having a first end and second end, wherein the first end comprises an inlet port;
a sleeve portion constructed substantially out of a semipermeable membrane, wherein the sleeve portion is disposed on an outer surface of the catheter body and is in fluid communication with the second end of the instillation lumen; and
a pump in fluid communication with the inlet port of the instillation lumen;
wherein the pump is operable to continuously move a fluid through the instillation lumen to the sleeve portion; and
wherein the sleeve portion is operable to continuously efflux said fluid through the semipermeable membrane.

35. The urinary catheter system of claim 34 further comprising a pressure and flow regulating valve attached to the instillation lumen, wherein the pressure and flow regulating valve is operable to control the flowrate of the fluid at specific pressures.

36. The urinary catheter system of claim 34 wherein the pump is an intravenous (IV) pump.

37. The urinary catheter system of claim 34 wherein the pump is a pump tension device.

38. A urinary catheter system comprising:
an elongate catheter body having a proximal end and a distal end, wherein the distal end of the elongate catheter body comprises a drainage opening operable to drain fluid from inside a patient's body and the proximal end of the elongate catheter body is operable to discharge said fluid from the catheter body;
a drainage lumen extending through the catheter body, wherein the drainage lumen communicates with the drainage opening and is operable to discharge fluid via the proximal end of the catheter body;
an instillation lumen disposed within and extending through the catheter body, wherein the instillation lumen has a first end and a second end, wherein the first end of the instillation lumen comprises an inlet port;
a sleeve portion constructed substantially out of a semipermeable membrane, wherein the sleeve portion is disposed on an outer surface of the catheter body and is in fluid communications with the second end of the instillation lumen; and
a pump in fluid communication with the inlet port of the instillation lumen;
wherein the pump is operable to continuously move a fluid through the instillation lumen to the sleeve portion; and
wherein the sleeve portion is operable to continuously efflux said fluid through the semipermeable membrane.

39. The urinary catheter system of claim 38 further comprising a pressure and flow regulating valve attached to the instillation lumen, wherein the pressure and flow regulating valve is operable to control the flowrate of the fluid at specific pressures.

40. The urinary catheter system of claim 38 wherein the pump is an intravenous (IV) pump.

41. The urinary catheter system of claim 38 wherein the pump is a pump tension device.

42. The urinary catheter system of claim 38 further comprising a retaining mechanism between the sleeve portion and the drainage opening of the drainage lumen.

43. A urinary catheter system comprising:
an elongate catheter body having a proximal end and a distal end;
a first instillation lumen disposed within and extending through the catheter body, the first instillation lumen having a first end and second end, wherein the first end comprises an inlet port;
a first sleeve portion constructed substantially out of a semipermeable membrane, wherein the first sleeve portion is disposed on an outer surface of the catheter body and is in fluid communication with the second end of the first instillation lumen;
a first pump in fluid communication with the inlet port of the first instillation lumen;
a second instillation lumen disposed within and extending through the catheter body, the second instillation lumen having a first end and second end, wherein the first end comprises an inlet port;
a second sleeve portion constructed substantially out of a semipermeable membrane, wherein the sleeve portion is disposed on an outer surface of the catheter and is in fluid communication with the second end of the second instillation lumen;
a second pump in fluid communication with the inlet port of the second instillation lumen;
wherein the first sleeve portion is closer to the proximal end of the elongate catheter body than the second sleeve portion;
wherein the first pump is operable to continuously move a first fluid through the first instillation lumen to the first sleeve portion;
wherein the first sleeve portion is operable to continuously efflux the first fluid through the semipermeable membrane;
wherein the second pump is operable to continuously move a second fluid through the second instillation lumen to the second sleeve portion; and
wherein the second sleeve portion is operable to continuously efflux the second fluid through the semipermeable membrane.

44. The urinary catheter system of claim 43 further comprising a pressure and flow regulating valve attached to the first instillation lumen, wherein the pressure and flow regulating valve is operable to control the flowrate of the first fluid within the first instillation lumen at specific pressures.

45. The urinary catheter system of claim 43 further comprising a pressure and flow regulating valve attached to the second instillation lumen, wherein the pressure and flow regulating valve is operable to control the flowrate of the second fluid within the second instillation lumen at specific pressures.

46. The urinary catheter system of claim 43 wherein either the first or second pump is an intravenous (IV) pump.

47. The urinary catheter system of claim 43 wherein either the first or second pump is a pump tension device.

48. The urinary catheter system of claim 43 further comprising a retaining mechanism between the first sleeve portion and the second sleeve portion.

49. The urinary catheter system of claim 43, wherein the distal end of the catheter body further comprises a drainage opening operable to drain fluid from inside a patient's body and the proximal end of the elongate catheter body is operable to discharge said fluid from the catheter body, wherein said urinary catheter system further comprises a drainage lumen disposed within and extending through the catheter body that communicates with the drainage opening and is operable to discharge fluid via the proximal end of the catheter body.

50. A method for cleaning the periurethral space while using a urinary catheter to drain the bladder, said method comprising:
providing a urinary catheter system comprising,
an elongate catheter body having a proximal end and a distal end, the elongate catheter body comprising one instillation lumen disposed within and extending through said catheter body, the instillation lumen having a first end and second end, wherein the first end comprises an inlet port;
a sleeve portion constructed substantially out of a semipermeable membrane, wherein the sleeve portion is disposed on an outer surface of the catheter body and is in fluid communication with the second end of the instillation lumen;
a pump in fluid communication with the inlet port of the instillation lumen;
wherein the pump is operable to continuously move a fluid through the instillation lumen to the sleeve portion; and
wherein the sleeve portion is operable to continuously efflux said fluid through the semipermeable membrane;
inserting the sleeve portion of the catheter body into the urethra of the patient;
pumping a fluid continuously through the instillation lumen to the sleeve portion; and
continuously effluxing fluid through the semipermeable membrane of the sleeve portion.

51. The method of claim 50 wherein the distal end of the catheter body within the urinary catheter system further comprises a drainage opening operable to drain fluid from inside a patient's body and the proximal end of the elongate catheter body is operable to discharge said fluid from the catheter body, wherein said urinary catheter system further comprises a drainage lumen disposed within and extending through the catheter body that communicates with the drainage opening and is operable to discharge fluid via the proximal end of the catheter body.

52. The method of claim 51 further comprising the step of inserting the drainage opening of the drainage lumen into the patient's bladder.

53. The method of claim 52 further comprising the step of plugging the entrance to the patient's bladder using a retaining mechanism positioned between the first sleeve portion and the drainage opening of the drainage lumen.
